(19) United States
(12) Patent Application Publication
(10) Pub. No.: US 2019/0117925 A1
(43) Pub. Date: Apr. 25, 2019

(54) HANDHELD POSITIVE AIRWAY PRESSURE MASK AND METHODS OF MAKING AND USING SAME

(71) Applicant: Duke University, Durham, NC (US)

(72) Inventor: Lindsay Boole, Durham, NC (US)

(21) Appl. No.: 16/167,046

(22) Filed: Oct. 22, 2018

Related U.S. Application Data
(60) Provisional application No. 62/575,644, filed on Oct. 23, 2017.

(51) Int. Cl. A61M 16/06; A61M 16/20

(52) U.S. Cl. CPC A61M 16/0616; A61M 16/08; A61M 16/208

(57) ABSTRACT

The present invention relates portable and hand-held devices having a facemask and a positive and expiratory pressure (PEEP) valve configured such that exhalation by a user into the device maintains the user's airway pressure at or above atmospheric pressure. The present invention further relates to methods of using such devices for purposes of ameliorating symptoms related to tracheobronchomalacia (TBM).

### CROSS-REFERENCE TO RELATED APPLICATIONS

The present application claims the priority benefit of U.S. Provisional Patent Application 62/575,644, filed Oct. 23, 2017, which is incorporated by reference in its entirety.

### FIELD OF INVENTION

The present invention relates to portable and hand-held devices having a facemask and a positive end expiratory pressure (PEEP) valve configured such that exhalation by a user into the device maintains the user's airway pressure at or above atmospheric pressure. The present invention further relates to methods of using such devices for purposes of ameliorating (e.g., relieving) symptoms related to tracheobronchomalacia (TBM).

### BACKGROUND

Patients suffering from tracheobronchomalacia and related symptoms (e.g., debilitating paroxysms of coughing, dyspnea, wheezing, and recurrent respiratory infections) can experience profound limitations on public excursions, loss of ability to work, recurrent hospital and even intensive care unit admissions or morbidity. Portable and handheld treatment options are quite limited. As such, there is a significant need for portable and handheld devices designed to assist patients suffering from tracheobronchomalacia and related symptoms (e.g., debilitating paroxysms of coughing, dyspnea, wheezing, and recurrent respiratory infections).

The present invention addresses this need.

### SUMMARY

The present invention relates portable and hand-held devices having a facemask and a positive end expiratory pressure (PEEP) valve configured such that exhalation by a user into the device maintains the user's airway pressure at or above atmospheric pressure. The present invention further relates to methods of using such devices for purposes of ameliorating (e.g., relieving) symptoms related to tracheobronchomalacia (TBM).

In certain embodiments, the present invention provides devices comprising a ventilation face mask and a positive end expiratory pressure (PEEP) valve, wherein the device is configured such that exhalation by a user into the device maintains the user's airway pressure at or above atmospheric pressure.

In some embodiments, the user is a human patient (e.g., adult human patient) (e.g., pediatric human patient). In some embodiments, the user is a human patient experiencing symptoms related to tracheobronchomalacia (TBM) (e.g., one or more of dyspnea, orthopnea, intractable cough, paroxysms of coughing, and wheezing).

Such devices are not limited to a specific configuration regarding the facemask and the PEEP valve. In some embodiments, the facemask has therein a ventilation port. In some embodiments, the PEEP valve is engaged with the facemask via insertion into the facemask port. In some embodiments, the PEEP valve is engaged with the facemask via insertion over the facemask port. In some embodiments, the PEEP valve is integrated with the facemask.

Such devices are not limited to a particular size of the facemask. In some embodiments, the size of the facemask is such that it can cover a user's mouth and nose.

Such devices are not limited to a particular type or kind of PEEP valve. In some embodiments, the PEEP valve is a one-way PEEP valve. In some embodiments, the PEEP valve is a two-way PEEP valve. In some embodiments, the amount of airflow resistance provided with the PEEP valve is adjustable. In some embodiments, the amount of airflow resistance provided with the PEEP valve is approximately 5 cm H₂O (e.g., 3, 3.1, 3.3, 3.5, 3.88, 3.95, 3.999, 4.1, 4.25, 4.4, 4.67, 4.85, 4.95, 4.99, 5, 5.01, 5.2, 5.3 5.4, 5.7, 5.85, 5.95, 5.99, 6.3, 6.25, etc.) to approximately 20 cm H₂O (e.g., 18, 18.1, 18.35, 18.75, 18.99, 19, 19.25, 19.36, 19.75, 19.88, 19.95, 19.999, 20, 20.001, 20.1, 20.23, 20.25, 20.8, 20.999, 21, 21.25, etc.).

In some embodiments, the devices further comprise an in-flow valve, wherein the in-flow valve permits a user to inhale while using the device. In some embodiments, the in-flow valve is positioned within or attached onto the facemask. In some embodiments, the in-flow valve is positioned within the PEEP valve.

In some embodiments, such devices are collapsible.

In certain embodiments, the present invention provides systems and/or kits comprising a device as described herein and instructions for use.

In certain embodiments, the present invention provides methods for treating a subject suffering from symptoms related to TBM, comprising having a user exhale (e.g., repeatedly exhale as needed) into a device as described herein. In such embodiments, the symptoms related to TBM include one or more of dyspnea, orthopnea, intractable cough, paroxysms of coughing, and wheezing.

In certain embodiments, the present invention provides methods for treating a subject suffering from symptoms related to softening of tracheal and bronchial cartilage, comprising having the subject exhale (e.g., repeatedly exhale as needed) into a device as described herein.

In certain embodiments, the present invention provides methods for treating a subject (e.g., an adult or pediatric human subject) suffering from symptoms related to dynamic narrowing of transverse or sagittal diameters of tracheobronchial lumen, comprising having the subject exhale (e.g., repeatedly exhale as needed) into a device as described herein.

In certain embodiments, the present invention provides methods for treating a subject (e.g., an adult or pediatric human subject) suffering from symptoms related to substantial invagination of the posterior membrane of trachebronchial tree (e.g., excessive dynamic airway collapse (EDAC)), comprising having the subject exhale (e.g., repeatedly exhale as needed) into a device as described herein.

In certain embodiments, the present invention provides methods for treating a subject (e.g., an adult or pediatric human subject) suffering from dyspnea, comprising having the subject exhale (e.g., repeatedly exhale as needed) into a device as described herein.

In certain embodiments, the present invention provides methods for treating a subject (e.g., an adult or pediatric human subject) suffering from orthopnea, comprising having the subject exhale (e.g., repeatedly exhale as needed) into a device as described herein.

In certain embodiments, the present invention provides methods for treating a subject (e.g., an adult or pediatric human subject) suffering from intractable cough, comprising having the subject exhale (e.g., repeatedly exhale as needed) into a device as described herein.

In certain embodiments, the present invention provides methods for treating a subject (e.g., an adult or pediatric human subject) suffering from paroxysms of coughing, comprising having the subject exhale (e.g., repeatedly exhale as needed) into a device as described herein.

In certain embodiments, the present invention provides methods for treating a subject (e.g., an adult or pediatric human subject) suffering from wheezing, comprising having the subject exhale (e.g., repeatedly exhale as needed) into a device as described herein.

In certain embodiments, the present invention provides a handheld positive airway pressure mask, the mask comprising: a flexible facemask in communication with a PEEP valve. In some embodiments, the PEEP valve comprises a pressure of about 5 to about 20 cm H₂O.

In certain embodiments, the present invention provides methods of decreasing the cough duration of a subject comprising: (a) placing the mask as described herein over the mouth of the subject such that a tight seal is formed; and (2) coughing or expiring into the mask until the coughing ceases. In some embodiments, the mask is held over the mouth of the subject using both hands. In some embodiments, the subject is suffering from TBM.

Additional embodiments are described herein.

### BRIEF DESCRIPTION OF THE DRAWINGS

FIG. 1 shows an embodiment of a device of the present invention. As shown, the device 1 has therein a facemask 2 with a facemask port 3. As shown, the facemask port 3 is engaged with a PEEP valve 4 such that the PEEP valve is inserted onto the facemask port 3.

FIG. 2 shows two human patients using a device of the present invention. As can be seen, the user is holding the device around the mouth/nose area such that an airtight seal is created. As can be seen, the facemask is placed over the nose and mouth. As can be seen the PEEP valve is engaged with the facemask via the facemask port.

FIG. 3 shows a device 1 having a facemask 2 engaged with a PEEP valve 4 via a facemask port 3. In addition, the device 1 further contains an in-flow valve 5. As shown, the in-flow valve 5 is positioned in line with the PEEP valve 4.

FIG. 4 shows a facemask 2 and facemask port 3 having a collapsible design. As shown, the facemask 2 and facemask port 3 having a collapsible design is shown placed within a device container 6.

### DETAILED DESCRIPTION

Tracheobronchomalacia (TBM) is a pathologic condition in which softening of tracheal and bronchial cartilage causes the dynamic narrowing of transverse or sagittal diameters of tracheobronchial lumen. Generally, this condition refers to a weakness of the trachea frequently due to a reduction and/or atrophy of the longitudinal elastic fibers of the pars membranacea or impaired cartilage integrity, such that the airway is softer and more susceptible to collapse (see, e.g., Johnson T H, et al., Radiology. 1973, 109: 576-580; Jokinen K, et al., Ann Clin Res. 1977, 9: 52-57). TBM may arise congenitally (from disorders associated with impaired cartilage maturation or in combination with other abnormalities like tracheoesopageal fistula) or it may be acquired from prior intubation, trauma, infections, long standing extrinsic compression, or chronic inflammation (see, e.g., Ikeda S, et al., Nihon Ronen Igakkai Zasshi. 1993, 30: 974-977; Jokinen K, et al., J. Otorhinolaryngol Relat Spec. 1976, 38: 178-186). Localized TBM is usually seen in patients with prolonged endotracheal intubation, tracheostomy, or vascular rings. The cause of diffuse, acquired TBM is often unknown, but it is frequently seen in patients with common respiratory conditions, such as chronic bronchitis.

This pathologic airway weakening can lead to a dynamic outflow obstruction associated with symptoms (e.g., dyspnea, orthopnea, intractable cough, and inability to clear secretions) which can sometimes mimic asthma or chronic bronchitis. Furthermore, TBM may also be associated with a substantial invagination of the posterior membrane of the tracheo-bronchial tree, an entity defined as excessive dynamic airway collapse (EDAC) (see, e.g., Murgu D S, et al., Pulm Perspectives. 2005, 22,3: 7-10).

TBM treatment options include silicone stenting to prevent tracheal constriction, surgery to strengthen or attempt to rebuild the walls, continuous positive airway pressure that has a machine blow small amounts of air into the trachea to keep it open (mainly at night) (e.g., a CPAP machine) or a tracheostomy to bypass the problematic part of the airway.

Absent from such treatment options are portable and handheld devices enabling a patient suffering from TBM to personally ameliorate (e.g., relieve) its symptoms without the need of either a CPAP machine or surgical intervention.

Experiments conducted during the course of developing embodiments for the present invention resulted in the development of devices (e.g., handheld positive airway pressure masks) designed to assist a patient in ameliorating symptoms related to TBM (e.g., dyspnea, orthopnea, intractable cough, paroxysms of coughing, wheezing, and inability to clear secretions). Indeed, such experiments determined that use of such devices by patients suffering from symptoms related to TBM experienced amelioration of such symptoms.

As such, the present invention provides easy to use and easy to carry devices (e.g., handheld positive airway pressure masks), related systems and related methods of use that solve the problem of enabling a patient to personally ameliorate symptoms related to TBM (e.g., dyspnea, orthopnea, intractable cough, paroxysms of coughing, wheezing, and inability to clear secretions) without the need for surgical intervention or a CPAP machine.

Such devices are not limited to a particular design. In certain embodiments, the design of the devices are such that the resulting device is capable of maintaining a user's (e.g., a patient experiencing symptoms of TBM) airway pressure at or above atmospheric pressure which thereby ameliorates (e.g., relieves) symptoms related to TBM (e.g., dyspnea, orthopnea, intractable cough, paroxysms of coughing, wheezing, and inability to clear secretions).

Lung volume at the end of exhalation is determined by the interplay between the elastic recoil of the lung (e.g., trying to collapse the lung) and the elastic recoil of the chest wall (e.g., acting to pull the lung open). Under normal circumstances these forces are in balance and the lung and airways do not collapse completely when they reach their smallest volume at end expiration.

TBM renders the airways more prone to collapse completely at the end of expiration. Spontaneously breathing patients may be able to overcome this to some extent by closing the glottis or pursing the lips and trapping air inside the airways at end expiration and by engaging their muscles of respiration.

In certain embodiments, the devices of the present invention are capable of maintaining a user's (e.g., a patient experiencing symptoms of TBM) airway pressure at or above atmospheric pressure which thereby ameliorates (e.g., relieves) symptoms related to TBM (e.g., dyspnea, orthopnea, intractable cough, paroxysms of coughing, wheezing, and inability to clear secretions) through use of a facemask engaged with a positive end expiratory pressure (PEEP) valve. In some embodiments, such devices are configured to increase the volume or pressure of gas inside the lungs and airways at the end of expiration and/or increase functional residual capacity (FRC) in physiological terms. In some embodiments, such devices are configured to retain a small amount of positive pressure in the airways at the end of expiration rather than letting them return to atmospheric pressure or even less, as can occur in TBM.

Such devices are not limited to a particular type or kind of facemask. In some embodiments, the facemask is any type or kind of mask that securely covers a patient's mouth. In some embodiments, the facemask is any type or kind of mask that covers a patient's nose and mouth in such way that an airtight seal is generated between the patient's mouth and the facemask. In some embodiments, the facemask is the same as or similar to a facemask found on a standard resuscitation bag.

Such devices are not limited to particular design or configuration of the facemask. In some embodiments, the facemask is any design or configuration that permits the resulting device capable of maintaining a user's (e.g., a patient experiencing symptoms of TBM) airway pressure at or above atmospheric pressure which thereby ameliorates (e.g., relieves) symptoms related to TBM (e.g., dyspnea, orthopnea, intractable cough, paroxysms of coughing, wheezing, and inability to clear secretions).

Such devices are not limited to a particular size for the facemask. In some embodiments, the size of the facemask is such that it can be used with any adult or pediatric patient.

Such devices are not limited to a particular manner of engagement between the facemask and the PEEP valve. In some embodiments, the facemask has therein a facemask port for engaging with the PEEP valve. Such embodiments are not limited to a particular manner of engagement between the facemask port and the PEEP valve. In some embodiments, engagement between the facemask port and the PEEP valve involves insertion of the PEEP valve onto the facemask port. In some embodiments, engagement between the facemask port and the PEEP valve involves insertion of the facemask port onto the PEEP valve.

Such embodiments are not limited to particular positioning or size of the facemask port. In some embodiments, the facemask port is positioned and sized such that it enables engagement with the PEEP valve in a way that renders the device capable of maintaining a user's (e.g., a patient experiencing symptoms of TBM) airway pressure at or above atmospheric pressure which thereby ameliorates (e.g., relieves) symptoms related to TBM (e.g., dyspnea, orthopnea, intractable cough, paroxysms of coughing, wheezing, and inability to clear secretions).

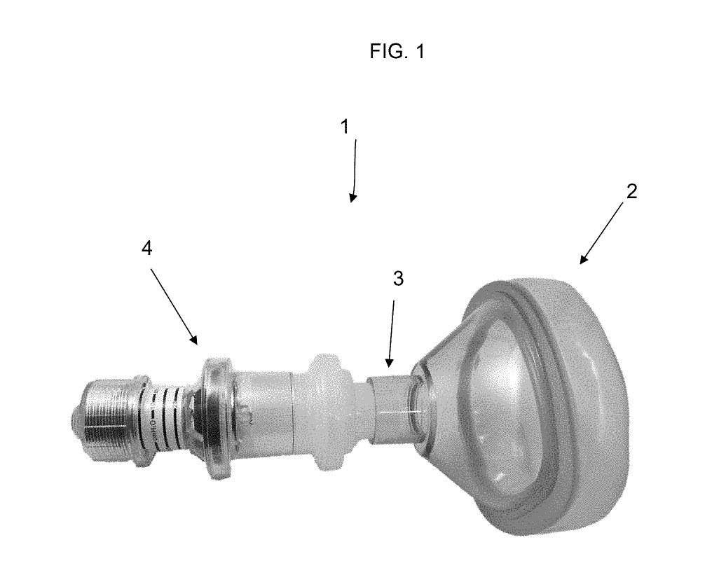

FIG. 1 shows an embodiment of a device of the present invention. As shown, the device 1 has therein a facemask 2 with a facemask port 3. As shown, the facemask port 3 is engaged with a PEEP valve 4 such that the PEEP valve is inserted onto the facemask port 3.

Such devices are not limited to a particular type or kind of PEEP valve. In some embodiments, the PEEP valve is configured to resist air exhaled by a user (e.g., a patient). In some embodiments, the PEEP valve is a one-way PEEP valve. In some embodiments, the PEEP valve is configured such that it can engage with the facemask via a facemask port. In some embodiments, the PEEP valve is configured to allow inhaled air to pass without resistance while providing a predetermined amount of resistance to air being exhaled from a user's lung. In some embodiments, the PEEP valve is of any design or configuration that permits the resulting device capable of maintaining a user's (e.g., a patient experiencing symptoms of TBM) airway pressure at or above atmospheric pressure which thereby ameliorates (e.g., relieves) symptoms related to TBM (e.g., dyspnea, orthopnea, intractable cough, paroxysms of coughing, wheezing, and inability to clear secretions). In some embodiments, the PEEP valve is any type or kind of commercially available PEEP valve.

Such PEEP valves are not limited to providing a certain predetermined amount of resistance to air being exhaled from a user's lung. In some embodiments, the predetermined amount of resistance to air being exhaled from a user's lung is between approximately 5 cm H₂O (e.g., 3, 3.1, 3.3, 3.5, 3.88, 3.95, 3.999, 4.1, 4.25, 4.4, 4.67, 4.85, 4.95, 4.99, 5, 5.01, 5.2, 5.3 5.4, 5.7, 5.85, 5.95, 5.99, 6.3, 6.25, etc.) to approximately 20 cm H₂O (e.g., 18, 18.1, 18.35, 18.75, 18.99, 19, 19.25, 19.36, 19.75, 19.88, 19.95, 19.999, 20, 20.001, 20.1, 20.23, 20.25, 20.8, 20.999, 21, 21.25, etc.). In some embodiments, the amount of resistance to air being exhaled from a user's lungs via the PEEP valve is adjustable.

Such PEEP valves are not limited to a particular construction. In some embodiments, the PEEP valve is constructed of silicone. In some embodiments, the PEEP valve contains a spring. In some embodiments, the PEEP valve has a flexible construction.

Referring against to FIG. 1, the PEEP valve 4 is shown engaged with the facemask 2 via the facemask port 3. As shown, the PEEP valve 4 is removable from the facemask 2 via disengagement with the facemask port 3.

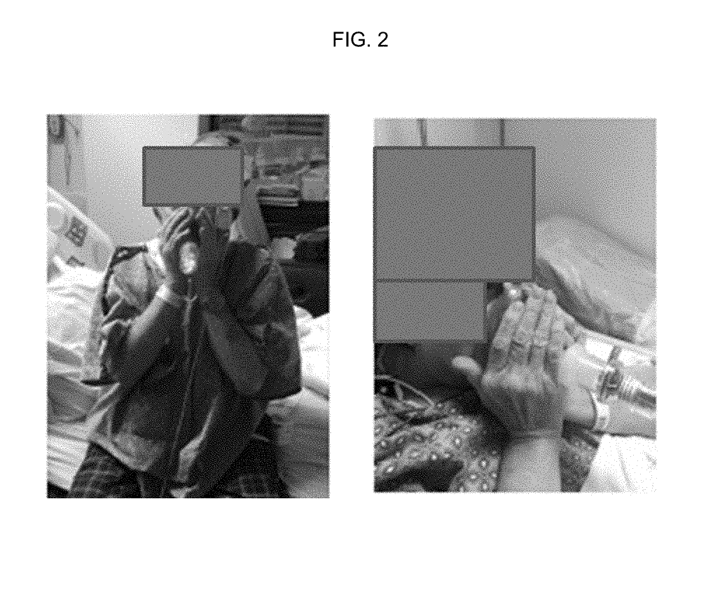

The devices of the present invention are not limited to particular manner of use by a user. In some embodiments, a user (e.g., a user experiencing symptoms related to TBM) engages the PEEP valve with the facemask via the facemask port. Next, a user places the device over the nose and mouth so as to create an airtight seal and exhales or coughs into the device. The user then removes the mask to permit inhalation. Indeed, FIG. 2 shows two human patients using a device of the present invention. As can be seen, the user is holding the device around the mouth/nose area such that an airtight seal is created. As can be seen, the facemask is placed over the nose and mouth. As can be seen the PEEP valve is engaged with the facemask via the facemask port.

In some embodiments, the PEEP valve is not removable but rather is integrated with the device. Such devices having an integrated PEEP valve could be overall smaller than devices having a removable PEEP valve. In such embodiments wherein the device contains a PEEP valve integrated with the facemask a facemask port would not be necessary.

Such devices of the present invention having a PEEP valve integrated with the facemask are not limited to particular manner of use by a user. In some embodiments, a user (e.g., a user experiencing symptoms related to TBM) places the device over the nose and mouth so as to create an airtight seal and exhales into the device. The user then removes the mask to permit inhalation.

In certain embodiments, the device further contains an in-flow valve for purposes of enabling a user to inhale without having to remove the device. Thus, for example, for patients suffering from a coughing fit such devices further having an in-flow valve thus removes the need to break the tight facial seal in order to take a breath during/after such coughing.

Such embodiments are not limited to a particular type, kind or size or positioning of the in-flow valve. In some embodiments, the in-flow valve is positioned in-line with the PEEP valve. In some embodiments, the in-flow valve is positioned separately from the PEEP valve, including directly within the facemask.

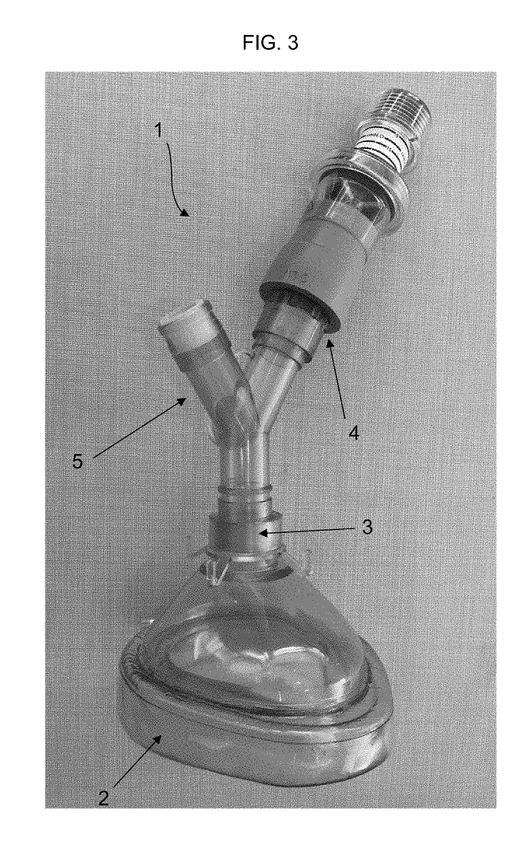

FIG. 3 shows a device 1 having a facemask 2 engaged with a PEEP valve 4 via a facemask port 3. In addition, the device 1 further contains an in-flow valve 5. As shown, the in-flow valve 5 is positioned in line with the PEEP valve 4.

Such devices of the present invention having an in-flow valve are not limited to particular manner of use by a user. In some embodiments, a user (e.g., a user experiencing symptoms related to TBM) engages the PEEP valve with the facemask via the facemask port (e.g., if the device has a PEEP valve not integrated with the facemask). Next, a user (e.g., a user experiencing symptoms related to TBM) places the device over the nose and mouth so as to create an airtight seal and exhales into the device causing the exhaled breath to exit via the PEEP valve. The user then inhales and air flows into the facemask via the in-flow valve. Exhalation and inhalation is thus repeated as necessary.

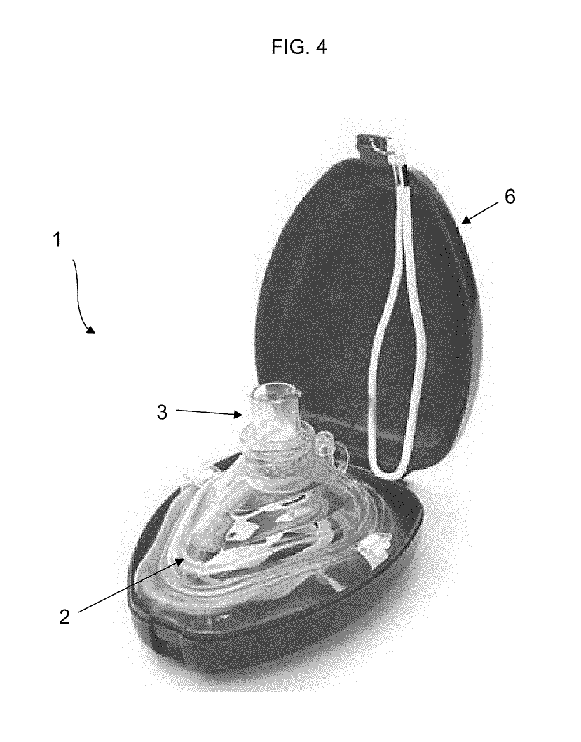

In certain embodiments, for purposes of improving portability, the device is collapsible. For example, in some embodiments, the design of the facemask is tiered or indented to allow for collapsibility, similar to a portable cardiopulmonary resuscitation (CPR) facemask. In such embodiments, such devices could then collapse, with or without requiring removal of a separate PEEP valve (depending on PEEP valve integration with the facemask). Indeed, FIG. 4 shows a facemask 2 and facemask port 3 having a collapsible design. As shown, the facemask 2 and facemask port 3 having a collapsible design is shown placed within a device container 6.

The devices of the present invention are not limited to particular manner of maintaining a user's (e.g., a patient experiencing symptoms of TBM) airway pressure at or above atmospheric pressure which thereby ameliorates (e.g., relieves) symptoms related to TBM (e.g., dyspnea, orthopnea, intractable cough, paroxysms of coughing, wheezing, and inability to clear secretions) through use of a facemask engaged with a PEEP valve. In some embodiments, use of such a device by a user suffering from symptoms of TBM prevents repetitive collapse and re-expansion of airways occurring with every breath.

In certain embodiments, kits and systems are provided containing one or more of the devices of the present invention and instructions for use. In some embodiments, kits and systems are provided containing a facemask having a facemask port and a separate PEEP valve and/or instructions for use. In some embodiments, kits and systems are provided containing a facemask having a facemask port and a separate PEEP valve containing an in-flow valve and/or instructions for use. In some embodiments, kits and systems are provided containing a facemask having an integrated PEEP valve and instructions for use.

In certain embodiments, the present invention provides methods for treating a subject suffering from symptoms related to TBM. Such methods involve, for example, placing the device over the subject's nose and mouth and having the subject exhale into the device. Such methods permit the user to maintain airway pressure at or above atmospheric pressure during exhalation (e.g., coughing) which thereby ameliorates (e.g., relieves) symptoms related to TBM (e.g., dyspnea, orthopnea, intractable cough, paroxysms of coughing, wheezing, and inability to clear secretions). Such methods prevent repetitive collapse and re-expansion of airways occurring during exhalation (e.g., coughing).

The devices of the present invention can be used for treating a wide variety of medical conditions. For example, the devices can be used for treating symptoms related to a softening of tracheal and bronchial cartilage. The devices can be used for treating symptoms related to a dynamic narrowing of transverse or sagittal diameters of tracheobronchial lumen. The devices can be used for treating symptoms related to substantial invagination of the posterior membrane of trachebronchial tree (e.g., excessive dynamic airway collapse (EDAC)). The devices can be used for treating dyspnea. The devices can be used for treating orthopnea. The devices can be used for treating intractable cough. The devices can be used for treating paroxysms of coughing. The devices can be used for treating wheezing.

### EXPERIMENTAL

### Example I

### 1. Case Presentation #1

70 yo male with COPD admitted to our institution for acute-on-chronic, severe cough, presumed to be COPD exacerbation; recently hospitalized×3 at another institution for similar presentation, including intubation for respiratory distress.
Recently quit his job at a retail store due to debility from coughing.
On exam, he had episodes of profound coughing paroxysms accompanied by respiratory distress with tachycardia and transient desaturation. The cough had a distinct vibratory quality.
Bronchoscopy revealed TBM with 100% expiratory collapse of a 9.5 cm segment of his trachea (FIG. 1a) and 90% collapse of the mainstem bronchi. Trachea measured>18 mm, too large for available stents.
No improvement with steroids, bronchodilators, antitussives, narcotics. CPAP aborted cough but he could not don equipment during episodes, given distress. Insurance denied CPAP for this indication.

### 2. Case Presentation #2

66 yo female with HTN, obesity, and GERD presenting to Interventional Pulmonology clinic with chronic cough.
Spirometry normal.
Failed inhaled bronchodilators, antitussives, and narcotics.
Bronchoscopy revealed TBM with 100% expiratory collapse of the mid and distal trachea (FIG. 1b) and bilateral mainstem bronchi.
Trachea measured 24 mm by CT scan, too large for available stents.

### 3. Parameters/Instructions for Subjects

The patients were taught to use both hands to hold the mask over their nose and mouth, forming a tight seal.
Mask was self-applied during coughing paroxysms and briefly removed for inhalation as needed, then replaced during coughing/exhalation.
PEEP valve was titrated to effect.
   15 cm H₂O was effective for patient #1
   7.5 cm H₂O was effective for patient #2
Coughing into the mask provided PEEP, pneumatically stenting the patients' airways and aborting coughing paroxysms.
Both patients experienced a subjective decline in cough duration and improvement in quality of life.

### 4. Conclusions

6 months after the device was introduced, patient #1 underwent tracheobronchoplasty with good result. He now has minimal residual cough, has been weaned off of all steroids (including inhaled corticosteroid), and is planning a return to work.
Patient #2 continues to use the device. She has been referred to thoracic surgery for consideration of tracheobronchoplasty, but states that she would like to avoid surgical intervention.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

We claim:

1. A device comprising a facemask and a positive and expiratory pressure (PEEP) valve, wherein the device is configured such that exhalation by a user into the device maintains the user's airway pressure at or above atmospheric pressure,
wherein the size of the facemask is such that it can cover a user's mouth and nose,
wherein the amount of airflow resistance provided with the PEEP valve is adjustable.

2. The device of claim 2, wherein the user is a human patient experiencing symptoms related to tracheobronchomalacia (TBM), wherein the symptoms of TBM are one or more of dyspnea, orthopnea, intractable cough, paroxysms of coughing, and wheezing.

3. The device of claim 1, wherein the facemask has therein a ventilation port.

4. The device of claim 1, wherein the PEEP valve is engaged with the facemask via insertion into the facemask port, or wherein the PEEP valve is engaged with the facemask via insertion over the facemask port, or wherein the PEEP valve is integrated with the facemask.

5. The device of claim 1, wherein the PEEP valve is a one-way PEEP valve, or wherein the PEEP valve is a two-way PEEP valve.

6. The device of claim 1, wherein the amount of airflow resistance provided with the PEEP valve is approximately 5 cm H₂O (e.g., 3, 3.1, 3.3, 3.5, 3.88, 3.95, 3.999, 4.1, 4.25, 4.4, 4.67, 4.85, 4.95, 4.99, 5, 5.01, 5.2, 5.3 5.4, 5.7, 5.85, 5.95, 5.99, 6.3, 6.25, etc.) to approximately 20 cm H₂O (e.g., 18, 18.1, 18.35, 18.75, 18.99, 19, 19.25, 19.36, 19.75, 19.88, 19.95, 19.999, 20, 20.001, 20.1, 20.23, 20.25, 20.8, 20.999, 21, 21.25, etc.).

7. The device of claim 1, wherein the device further comprises an in-flow valve, wherein the in-flow valve permits a user to inhale while using the device.

8. The device of claim 7, wherein the in-flow valve is positioned within or attached onto the facemask, or wherein the in-flow valve is positioned within the PEEP valve.

9. The device of claim 1, wherein the device is collapsible.

10. A kit comprising a device of claim 1 and instructions for use.

11. A method for treating a subject, comprising having the subject exhale into a device of claim 1, wherein the subject is suffering from one or more of symptoms related to TBM, symptoms related to softening of tracheal and bronchial cartilage, symptoms related to dynamic narrowing of transverse or sagittal diameters of tracheobronchial lumen, symptoms related to substantial invagination of the posterior membrane of trachebronchial tree (e.g., excessive dynamic airway collapse (EDAC)), dyspnea, orthopnea, intractable cough, paroxysms of coughing, and wheezing.

12. The method of claim 11, wherein the subject is a human subject.

13. The method of claim 12, wherein the human subject is suffering from TBM.

14. The method of claim 11, wherein the subject exhales into the device once or repeatedly as needed.
